(19) United States
(12) Patent Application Publication
(10) Pub. No.: US 2024/0000594 A1
(43) Pub. Date: Jan. 4, 2024

(54) INTEGRATED DEPLOYMENT BALLOON STENT DELIVERY

(71) Applicant: Merit Medical Systems, Inc., South Jordan, UT (US)

(72) Inventors: John Hall, Bountiful, UT (US); Wayne Mower, Bountiful, UT (US); Christopher Cindrich, Highland, UT (US)

(21) Appl. No.: 18/342,915

(22) Filed: Jun. 28, 2023

Related U.S. Application Data
(60) Provisional application No. 63/367,411, filed on Jun. 30, 2022.

(51) Int. Cl. A61F 2/97; A61F 2/95

(52) U.S. Cl. CPC A61F 2/97; A61F 2/9517; A61F 2002/9665

(57) ABSTRACT

A vascular stent deployment device and related methods are disclosed. In some embodiments the deployment device may include a delivery catheter assembly. The delivery catheter assembly may include a self-expanding stent constrained by a rupturable sleeve. The self-expanding stent is disposed around an expandable member configured to partially expand to rupture the sleeve allowing the self-expanding stent to deploy. The expandable member may also be configured to aid in proximal displacement of the self-expanding stent as the delivery catheter assembly is manipulated to deploy the self-expanding stent.

### RELATED CASES

This application claims priority to U.S. Provisional Application No. 63/367,411, filed on Jun. 30, 2022 and titled “INTEGRATED DEPLOYMENT BALLOON STENT DELIVERY,” which is hereby incorporated by reference in its entirety.

### TECHNICAL FIELD

The present disclosure relates generally to medical devices. More specifically, the present disclosure relates to a vascular prosthesis deployment device and a method of deploying an expandable vascular prosthesis. In some embodiments, the present disclosure relates to a vascular prosthesis deployment device and a method used to control radial expansion of an expandable vascular prosthesis during deployment.

### BRIEF DESCRIPTION OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1 is a perspective view of a deployment device.

FIG. 2 is a cross-sectional view of a portion of the deployment device of FIG. 1.

FIG. 3A is a perspective view of a ratchet slide component of the deployment device of FIGS. 1 and 2.

FIG. 3B is a cross-sectional view of the ratchet slide of FIG. 3A.

FIG. 4 is a side view of a carrier component of the deployment device of FIGS. 1 and 2.

FIG. 5 is a cross-sectional view of another portion of the deployment device shown in FIGS. 1 and 2.

FIG. 6 is a cross-sectional view of yet another portion of the deployment device shown in FIGS. 1 and 2.

FIG. 7 is a front view of the deployment device of FIG. 1, illustrating certain cross-sectional planes described herein.

FIG. 8 is a perspective view of the safety member of the deployment device of FIG. 1.

FIG. 9 is a side view of a distal portion of a delivery catheter assembly of the deployment device of FIG. 1.

FIG. 10A is a side view of the distal portion of the delivery catheter assembly of the deployment device of FIG. 1.

FIG. 10B is a longitudinal cross-sectional view of the distal portion of the delivery catheter assembly of FIG. 10A along plane 10B-10B.

FIG. 10C is a transverse cross-sectional view of the portion of the delivery catheter assembly of FIG. 10A along plane 10C-10C.

FIG. 11A is a side view of the distal portion of the delivery catheter assembly of the deployment device of FIG. 1 in a first state of stent deployment.

FIG. 11B is a side view of the distal portion of the delivery catheter assembly of FIG. 11A in a second state of prosthesis deployment.

FIG. 11C is a side view of the distal portion of the delivery catheter assembly of FIG. 11A in a third state of prosthesis deployment.

FIG. 11D is a side view of the portion of the delivery catheter assembly of FIG. 11A in a fourth state of prosthesis deployment.

FIG. 11E is a side view of the portion of the delivery catheter assembly of FIG. 11A in a fifth state of prosthesis deployment.

FIG. 11F is a side view of the fully deployed and expanded stent.

### DETAILED DESCRIPTION

Deployment devices may be configured to deliver a medical appliance to a location within a patient's body and deploy the medical appliance within the patient's body. Though specific examples recited herein may refer to deployment of devices within the vasculature, analogous concepts and devices may be used in various other locations within the body, including for placement and deployment of medical appliances in the gastrointestinal tract (including, for example, within the esophagus, intestines, stomach, small bowel, colon, and biliary duct), the respiratory system (including, for example, within the trachea, bronchial tubes, lungs, nasal passages, and sinuses), or any other location within the body, both within bodily lumens (for example, the ureter, the urethra, and/or any of the lumens discussed above) and within other bodily structures.

Furthermore, though specific examples herein may refer to deployment of vascular prostheses such as stents, deployment of a wide variety of medical appliances are within the scope of this disclosure, including stents, stent-grafts, shunts, grafts, and so forth. The deployment device disclosed herein may be configured to deliver and deploy self-expanding medical appliances, including stents configured to self-expand within a bodily lumen upon deployment. Additionally, the deployment device disclosed herein may be configured to further expand or otherwise place a self-expanding stent, via deployment of a balloon, to maximize a diameter of the self-expanding stent and to smooth the self-expanding stent of wrinkles. A benefit of stent diameter maximization is a maximized diameter of a lesion within the bodily lumen beyond the ability of the self-expanding stent that allows for unrestricted fluid flow through the bodily lumen. A benefit of smoothing the stent is to prevent turbulent fluid flow (e.g., blood flow) that may cause thrombus formation within the stent.

As used herein, delivery of a medical appliance generally refers to placement of a medical appliance in the body, including displacement of the appliance along a bodily lumen to a treatment site. For example, delivery includes displacement of a self-expanding stent along a vascular lumen from an insertion site to a treatment location. Deployment of a medical appliance refers to placement of the medical appliance within the body such that the medical appliance interacts with the body at the point of treatment. For example, deployment includes releasing a crimped or otherwise constrained self-expandable stent from a deployment device such that the stent is expanded and contacts a lumen of the vasculature. The deployment may include controlled partial expansion of a balloon to rupture a sleeve surrounding the constrained self-expanding stent and full expansion of the balloon to fully expand and smooth the self-expanding stent.

Embodiments of deployment devices within the scope of this disclosure include a handle assembly and a delivery catheter assembly. The handle assembly can include a housing and an actuator. The delivery catheter assembly can include an outer sheath operably coupled to the actuator and an inner sheath coupled to the housing. In certain embodiments, an intermediate sheath is coupled to the housing and disposed between the outer sheath and the inner sheath. The delivery catheter assembly may further include a pliant expandable member (e.g., balloon) surrounding a distal portion of the inner sheath, a self-expanding stent surrounding the balloon, and a rupturable sleeve surrounding and constraining the self-expanding stent. In some embodiments, the balloon, the self-expanding stent, and the sleeve are disposed in an annular space defined by the outer sheath and the inner sheath.

Embodiments of methods of deployment of the self-expanding stent may include steps of positioning a distal portion of the delivery catheter assembly at a lesion of a treatment site, withdrawing or retracting the outer sheath with the actuator, partially expanding the balloon to rupture the sleeve, self-expanding the self-expanding stent, and fully expanding the balloon to fully expand and to smooth the self-expanding stent.

Components of the embodiments as generally described and illustrated in the figures herein could be arranged and designed in a wide variety of configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Again, though the embodiments specifically described below may reference a vascular stent deployment device specifically, the concepts, devices, and assemblies discussed below may be analogously applied to deployment of a wide variety of medical appliances in a wide variety of locations within the body.

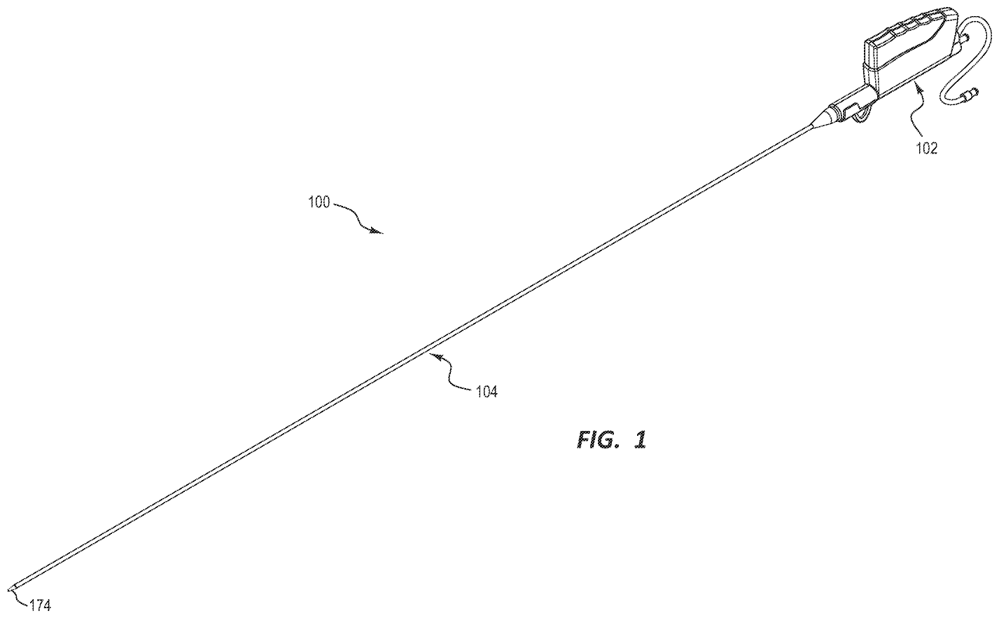

FIG. 1 is a perspective view of a deployment device 100. In the illustrated embodiment, the deployment device 100 comprises a handle assembly 102 adjacent the proximal end of the deployment device 100. An elongate delivery catheter assembly 104 extends distally from the handle assembly 102 to a distal tip or delivery tip 174. The handle assembly 102 may provide a proximal user input, with one or more components configured to allow a practitioner to deploy or otherwise manipulate a stent disposed within the delivery catheter assembly 104.

In use, the handle assembly 102 may be disposed outside of a patient's body, while the delivery catheter assembly 104 is advanced to a treatment location within the patient's body. For example, the delivery catheter assembly 104 may be advanced from an insertion site (such as, for example, a femoral or jugular insertion site) to a treatment location within the vasculature. As further detailed below, the delivery catheter assembly 104 may be configured to be advanced through bends, turns, or other structures within the anatomy of the vasculature. Again, as detailed below, a stent may be disposed within a portion of the delivery catheter assembly 104 such that a practitioner may deploy the stent from a distal end of the delivery catheter assembly 104 through manipulation of one or more components of the handle assembly 102.

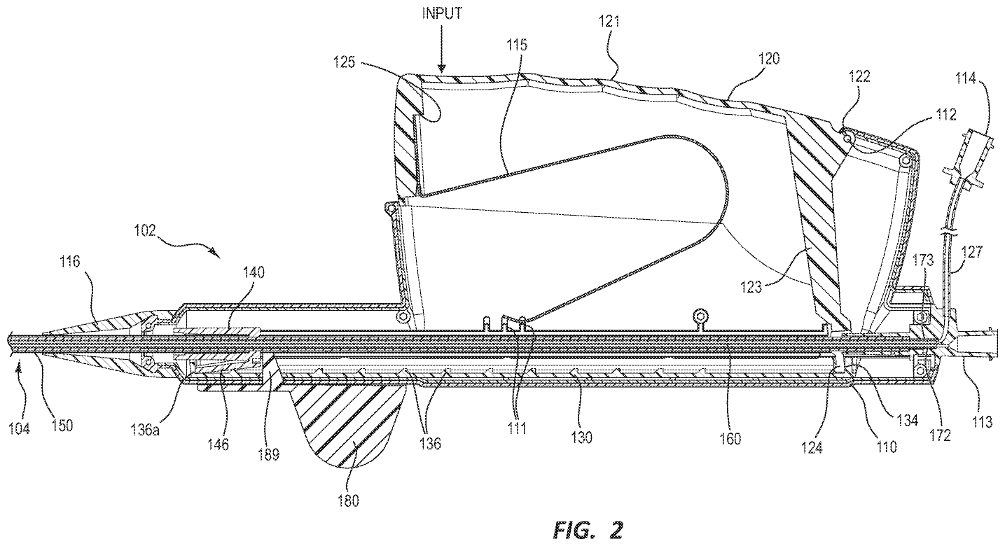

FIG. 2 is a cross-sectional view of a portion of the deployment device 100 of FIG. 1. Specifically, FIG. 2 is a side view of a portion of the deployment device 100 of FIG. 1, taken through a cross-sectional plane extending vertically and intersecting a longitudinal axis of the deployment device 100, when the deployment device 100 is positioned as shown in FIG. 1. The longitudinal axis of the deployment device 100 extends along the center of the delivery catheter assembly 104, including along the center of components of the delivery catheter assembly 104 that overlap with the handle assembly 102, such as an intermediate sheath 160, as shown in FIG.

As the handle assembly 102 is configured to be grasped or otherwise manipulated by a user and the delivery catheter assembly 104 is configured to extend to a treatment location within a patient's body, along the longitudinal axis, the delivery catheter assembly 104 extends in a distal direction away from the handle assembly 102. The proximal direction is opposite, correlating to a direction defined along the longitudinal axis, extending from the distal tip 174 toward the handle assembly 102.

FIG. 2 depicts various internal components of the handle assembly 102, exposed by the cross-sectional view. A portion of the delivery catheter assembly 104 is also shown extending from the handle assembly 102. The handle assembly 102 comprises a housing 110. The housing 110 surrounds certain components of the handle assembly 102, as shown, providing a grip surface for a practitioner.

The housing 110 is operably coupled to an actuator 120. Manipulation of the actuator 120 with respect to the housing 110 may be configured to deploy the stent, as further detailed below. In the depicted embodiment, the actuator 120 is rotatably coupled to the housing 110 by a pin 112. The pin 112 extends from the housing 110 and may be integrally formed with one or more other portions of the housing 110. As shown, the pin 112 extends through a pin aperture 122 in the actuator 120.

Other arrangements for operably coupling the actuator 120 and the housing 110 are within the scope of this disclosure. For example, the pin 112 may be integral with a portion of the actuator 120 and may be received in an opening, sleeve, or aperture formed in the housing 110. Other types of designs of rotatable couplings, including a separate coupling component such as a hinge, are within the scope of this disclosure. Still further, a compliant mechanism, such as a deformable flange, may be utilized to rotatably couple the actuator 120 and the housing 110, including compliant couplings integrally formed with the actuator 120, the housing 110, or both. Moreover, it is within the scope of this disclosure to slidably couple an actuator (such as actuator 120) to a housing (such as housing 110). Configurations wherein the actuator 120 is manipulated through rotation, translation, or other displacement relative to the housing 110 are all within the scope of this disclosure.

The actuator 120 comprises an input portion 121 extending from the pin aperture 122. In the depicted embodiment, the input portion 121 comprises a surface, at least partially exposed with respect to the housing 110. In operation, a user may manipulate the actuator 120 by exerting a force on the input portion 121, illustrated by the arrow labeled “input” in FIG. 2, displacing the input portion 121 generally toward the longitudinal axis of the deployment device (100 of FIG. 1) and causing the actuator 120 to rotate about the pin 112 with respect to the housing 110. Displacement of the actuator 120 due to a force such as illustrated by the arrow labeled “input” corresponds to “depression” of the actuator 120 or “depression of the actuator 120 with respect to the housing 110.”

The actuator 120 may further comprise a transfer arm 123 extending from the pin aperture 122. The transfer arm 123 may be rigidly coupled to the input portion 121, including embodiments wherein both the transfer arm 123 and the input portion 121 are integrally formed with the rest of the actuator 120. The transfer arm 123 extends to a ratchet slide engaging portion 124. Depression of the input portion 121, in the direction shown by the arrow labeled “input,” displaces the transfer arm 123 as the actuator 120 is rotated about the pin 112.

Depression of the input portion 121 thus causes displacement of the ratchet slide engaging portion 124 with respect to the housing 110. This displacement of the ratchet slide engaging portion 124 can be understood as rotation about the pin 112 having a proximal translation component and a vertical translation component, as rotation of the input portion 121 in the direction indicated by the arrow labeled “input” will displace (with respect to the housing 110) the ratchet slide engaging portion 124 both proximally and vertically.

A leaf spring 115 may be disposed between the actuator 120 and the housing 110. The spring 115 may be configured to resist displacement of the actuator 120 in the direction indicated by the arrow labeled “input” and may be configured to return the actuator 120 to the relative position shown in FIG. 2 after it has been depressed by a user. When the handle assembly 102 is unconstrained, the spring 115 may thus maintain (or return to) the relative position of the actuator 120 with respect to the housing 110 as shown in FIG. 2.

In the illustrated embodiment, the spring 115 engages with a spring ledge 125 of the actuator 120 and spring protrusions 111 of the housing 110. The spring protrusions 111 may provide a bearing surface for the spring 115 offset from movable internal components of the handle assembly 102 (such as a carrier 140 further detailed below). Though three spring protrusions 111 are shown in the depicted embodiment, more or fewer protrusions, or use of other features such as ridges, ledges, shoulders, and so forth, are within the scope of this disclosure.

The depicted embodiment comprises the spring 115. Other biasing elements, such as coil springs, piston assemblies, compliant mechanisms, and so forth, are likewise within the scope of this disclosure. In some instances, a compliant portion of one or both of the housing 110 and the actuator 120 may provide a biasing force analogous to that provided by the spring 115. Leaf springs, such as spring 115, may be configured to provide a relatively constant biasing force notwithstanding compression of the spring 115 as the actuator 120 is rotated or depressed with respect to the housing 110.

As the actuator 120 is depressed with respect to the housing 110, the spring 115 compresses and the ratchet slide engaging portion 124 is displaced as described above. Again, the displacement of the ratchet slide engaging portion 124 with respect to the housing 110 can be understood as having a proximal component and a vertical component.

The ratchet slide engaging portion 124 may be operably coupled to a ratchet slide 130 such that displacement of the ratchet slide engaging portion 124 likewise displaces the ratchet slide 130. The ratchet slide 130 may be constrained such that the ratchet slide 130 is configured only for proximal or distal displacement with respect to the housing 110. Thus, operable coupling of the ratchet slide engaging portion 124 to the ratchet slide 130 may allow for sliding interaction between the ratchet slide engaging portion 124 and the ratchet slide 130 such that only the proximal or distal component of the displacement of the ratchet slide engaging portion 124 is transferred to the ratchet slide 130. Stated another way, the ratchet slide 130 may be displaced in a direction parallel to the longitudinal axis of the deployment device 100 while the input displacement may be at an angle to the longitudinal axis of the deployment device 100. It is noted that, in the configuration shown in FIG. 2, a safety member 180 may prevent proximal displacement of the ratchet slide 130. The safety member 180, including removal thereof, is discussed in more detail below. Discussion herein relating to displacement of the ratchet slide 130 and related components may thus be understood as disclosure relevant to a configuration of the handle assembly 102 in which the safety member 180 has been removed.

As the actuator 120 is depressed with respect to the housing 110, the ratchet slide 130 may thus be proximally displaced with respect to the housing 110. One or both of the ratchet slide 130 and the actuator 120 may also interact with the housing 110 such that there is a positive stop to arrest the depression of the actuator 120 and/or proximal displacement of the ratchet slide 130. This positive stop may be an engaging ledge, shoulder, lug, detent, or other feature coupled to the housing 110, including features integrally formed on the housing 110.

A full stroke of the actuator 120 may thus correspond to displacement from the unconstrained position shown in FIG. 2 to the positive stop caused by interaction with the housing 110 when the actuator 120 is depressed. Release of the actuator 120 following a full or a partial stroke may then result in a return of the actuator 120 to the unconstrained state, due to the biasing force provided by the spring 115. The unconstrained state shown in FIG. 2 refers to lack of constraint due to user input. In this state, the spring 115 may be partially compressed, and interaction between the actuator 120 and the housing 110 may prevent rotation of the actuator 120 about the pin 112 in the opposite direction to depression of the actuator 120, or the return direction. In other words, interaction between the actuator 120 and the housing 110 (or features of the housing 110) may create a positive stop to the return motion of the actuator 120 as well.

Referring to both FIGS. 1 and 2, the actuator 120 and the housing 110 may be coupled such that pinching of external materials (such as a practitioner's hand or a surgical drape) is minimized when the actuator 120 is depressed or returned. For instance, the actuator 120 may comprise a shell configured to mate with, and slide into, the housing 110. Though the components may slide and rotate with respect to each other, the interface of the components may be sufficiently close and/or smooth to minimize pinching or other engagement of external materials. This close and/or smooth interface may refer to interaction at the edges of the actuator 120 as it is displaced into the housing 110 and/or to interaction at the portion of the actuator 120 near the pin 112, as the actuator 120 returns to the unconstrained position.

As also shown in FIGS. 1 and 2, the input portion 121 of the actuator 120 may also comprise ridges or other features to facilitate handling or gripping of the actuator 120 during use.

Referring again to FIG. 2, the ratchet slide 130 may thus be proximally displaced during depression of the actuator 120. Again, such displacement may correspond to a configuration in which the safety member 180 shown in FIG. 2 has been removed. Proximal displacement of the ratchet slide 130 may also proximally displace the carrier 140 due to interaction between one or more carrier engaging ratchet lugs 136 on the ratchet slide 130 and a ratchet slide engaging arm 146 coupled to the carrier 140.

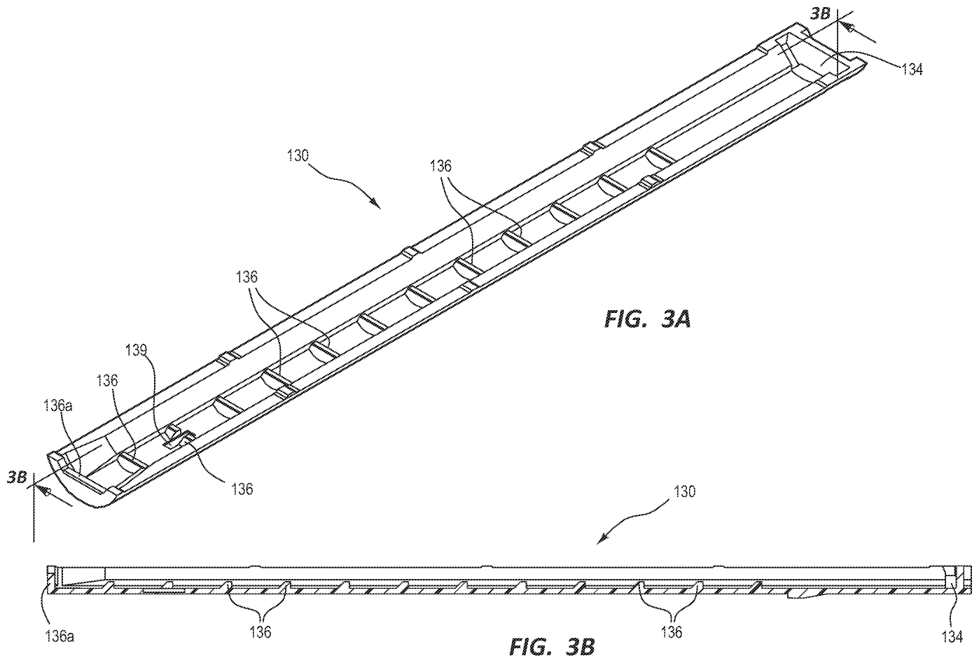

FIG. 3A is a perspective view of the ratchet slide 130 of the deployment device 100 of FIGS. 1 and 2. FIG. 3B is a cross-sectional view of the ratchet slide 130 of FIG. 3A, taken through a vertical plane disposed along a longitudinal centerline of the ratchet slide 130. When the ratchet slide 130 is disposed within the handle assembly 102 of FIG. 2, this cross-sectional plane would intersect the longitudinal axis of the deployment device 100.

As shown in FIGS. 2, 3A, and 3B, the ratchet slide 130 may comprise a plurality of carrier engaging ratchet lugs 136. The carrier engaging ratchet lugs 136 may be spaced at even intervals along the longitudinal direction of the ratchet slide 130. In the figures, exemplary carrier engaging ratchet lugs are denoted by reference numeral 136, while the distal most carrier engaging ratchet lug, disposed at the distal end of the ratchet slide 130, is denoted by reference numeral 136a.

The ratchet slide 130 further comprises a ratchet slide safety opening 139 and an actuator engaging opening 134. These features are discussed in more detail below.

As noted above, interaction between the ratchet slide engaging portion 124 of the actuator 120 and the ratchet slide 130 may proximally displace the ratchet slide 130 with respect to the housing 110. Engagement between the carrier 140 and one of the carrier engaging ratchet lugs 136 may also proximally displace the carrier 140 as the ratchet slide 130 is proximally displaced with respect to the housing 110. In the configuration of FIG. 2, the ratchet slide engaging arm 146 of the carrier 140 is engaged with the distal most carrier engaging ratchet lug 136a.

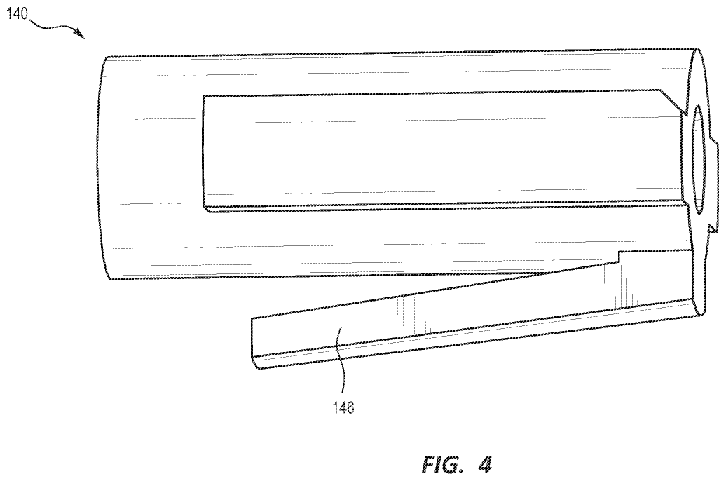

FIG. 4 is a side view of the carrier 140 of the deployment device 100 of FIGS. 1 and 2. As shown in FIG. 4, the ratchet slide engaging arm 146 extends radially away from a longitudinal axis of the carrier 140. When the carrier 140 is disposed within the handle assembly 102 of FIG. 2, the longitudinal axis of the carrier 140 is disposed along the longitudinal axis of the deployment device 100.

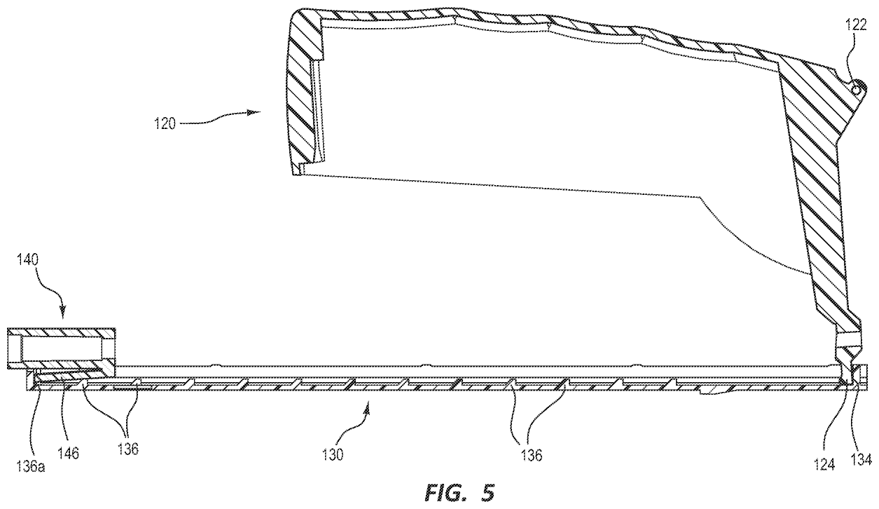

FIG. 5 is a cross-sectional view of a portion of the deployment device 100 shown in FIGS. 1 and 2. Specifically, the actuator 120, the ratchet slide 130, and the carrier 140 are shown in FIG. 5, in the same relative positions, and along the same cross-sectional plane as in FIG. 2.

Referring to FIGS. 2-5, during depression of the actuator 120 with respect to the housing 110, the actuator 120 rotates around the pin aperture 122. This rotation causes displacement of the ratchet slide engaging portion 124 of the actuator 120. The component of this displacement correlating to proximal displacement of the ratchet slide engaging portion 124 also proximally translates the ratchet slide 130 due to interaction between the ratchet slide engaging portion 124 of the actuator 120 and the actuator engaging opening 134 of the ratchet slide 130. Stated another way, the walls or faces that define the actuator engaging opening 134 may contact the ratchet slide engaging portion 124 such that the ratchet slide 130 is displaced when the actuator 120 is displaced.

Proximal displacement of the ratchet slide 130 also proximally displaces the carrier 140 due to interaction between the carrier engaging ratchet lugs 136 and the ratchet slide engaging arm 146. In the depicted embodiment, a distal surface of the ratchet slide engaging arm 146 is in contact with a proximal face of the distal most carrier engaging ratchet lug 136a. This contact exerts proximal force on the distal surface of the ratchet slide engaging arm 146, displacing the carrier 140 in a proximal direction. Accordingly, the ratchet slide 130 and the carrier 140 will move proximally until the actuator 120 reaches the end of the stroke.

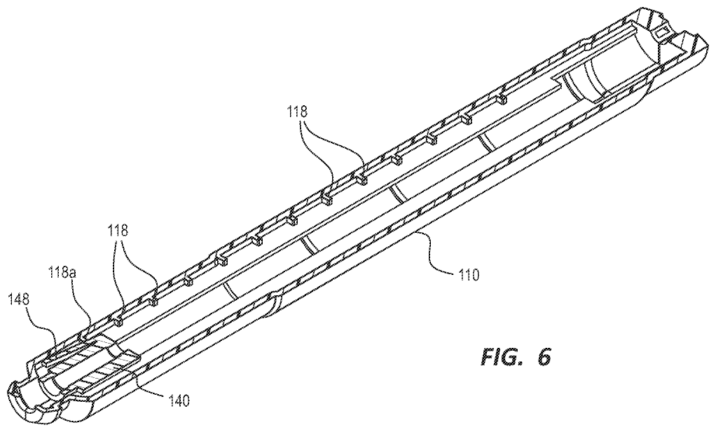

FIG. 6 is a cross-sectional view of the housing 110 and the carrier 140 in the same relative positions shown in FIG. 2. The cross-sectional plane of FIG. 6 extends along the longitudinal axis of the deployment device 100; however, the cross-sectional plane of FIG. 6 extends horizontally, orthogonal to the cross-sectional planes of FIGS. 2, 3B, and 5.

As shown in FIG. 6, the carrier 140 comprises a housing engaging arm 148 extending radially away from a longitudinal axis of the carrier 140. The housing 110 comprises a plurality of carrier engaging housing lugs 118. In FIG. 6, exemplary carrier engaging housing lugs are denoted by reference numeral 118, with the distal most carrier engaging housing lug denoted by reference numeral 118a.

Referring to FIGS. 2-6, as interaction between the actuator 120, the ratchet slide 130, and the carrier 140 displaces the carrier 140 with respect to the housing 110 (as shown and described above), the housing engaging arm 148 (shown in FIG. 6) of the carrier 140 will deflect radially inward due to contact with one of the carrier engaging housing lugs 118. For example, from the position shown in FIG. 6, as interaction between the distal most carrier engaging ratchet lug 136a and the ratchet slide engaging arm 146 of the carrier 140 draws the carrier 140 proximally, the distal most carrier engaging housing lug 118a causes the housing engaging arm 148 to displace radially inward. The housing engaging arm 148 will continue to deflect radially inward until the distal end of the housing engaging arm 148 is positioned proximal of the distal most carrier engaging housing lug 118a, at which point the housing engaging arm 148 will return to the radially outward configuration shown in FIG. 6. The point at which the housing engaging arm 148 moves proximally of the distal most carrier engaging housing lug 118a may correspond to the stroke of the actuator 120, such that engagement between the housing engaging arm 148 and the next carrier engaging housing lug 118 (moving in a proximal direction) occurs at the end of the stroke, which may correspond to contact between the ratchet slide 130 and/or the actuator 120 and a positive stop on the housing 110 defining the end of the stroke.

As the actuator 120 is released following the stroke, interaction between the spring 115, the housing 110, and the actuator 120 will return the actuator 120 to the unconstrained position (the position shown in FIG. 2) as discussed above. Corresponding rotation of the actuator 120 about the pin aperture 122 will thus correlate to displacement of the ratchet slide engaging portion 124, including a component of displacement in the distal direction. Interaction between the ratchet slide engaging portion 124 and the actuator engaging opening 134 will then correlate to distal displacement of the ratchet slide 130. Thus, when the actuator 120 is released at the end of a stroke, the actuator 120, the spring 115, and the ratchet slide 130 return to the same positions relative to the housing as shown in FIG. 2.

As the actuator 120 returns to the unconstrained position, however, interaction between the housing engaging arm 148 and the carrier engaging housing lug 118 prevents distal displacement of the carrier 140. Specifically, the distal surface of the housing engaging arm 148 will be in contact with a proximal facing surface of a carrier engaging housing lug 118, the interaction preventing the carrier 140 from returning to the pre-stroke position. In the exemplary stroke discussed above, the distal most carrier engaging housing lug 118a displaces the housing engaging arm 148 during the stroke, and the housing engaging arm 148 engages with the distal most carrier engaging housing lug 118a following the stroke. Subsequent strokes move the carrier 140 along the plurality of carrier engaging housing lugs 118 in a proximal direction.

As the actuator 120 returns to the unconstrained state, radially inward displacement of the ratchet slide engaging arm 146 of the carrier 140 allows the ratchet slide 130 to move distally with respect to the carrier 140, as engagement between the carrier 140 and the carrier engaging housing lugs 118 arrest distal displacement of the carrier 140.

Referring to FIGS. 2-6, with particular reference to the view of FIG. 5, distal displacement of the ratchet slide 130 with respect to the carrier 140 creates interaction between the carrier engaging ratchet lugs 136 and the ratchet slide engaging arm 146 causing the ratchet slide engaging arm 146 to displace radially inward. The proximal facing surface of the carrier engaging ratchet lugs 136 may be angled to facilitate this interaction. In the exemplary stroke discussed above, engagement between the distal most carrier engaging ratchet lug 136a displaces the carrier 140 in a proximal direction; during the return of the actuator 120, the next carrier engaging ratchet lug 136 (in a proximal direction) causes the radially inward displacement of the ratchet slide engaging arm 146 until the ratchet slide engaging arm 146 is proximal of the carrier engaging ratchet lug 136. At that point the ratchet slide engaging arm 146 returns to a radially outward position (analogous to that shown in FIG. 5) though the distal surface of the ratchet slide engaging arm 146 is now engaged with a proximal face of the next carrier engaging ratchet lug 136 (again in a proximal direction). Displacement of the ratchet slide 130 sufficient to move to engagement with a subsequent carrier engaging ratchet lug 136 may correspond with the magnitude of ratchet slide 130 displacement corresponding to a return of the actuator 120. Subsequent returns of the actuator 120 following strokes move the ratchet slide 130 such that the plurality of carrier engaging ratchet lugs 136 may serially engage the carrier 140, stroke after stroke.

Accordingly, as described above, depressing the actuator 120 for a full stroke, then allowing the actuator 120 to return to the unconstrained position, displaces the carrier 140 with respect to the housing 110 in discrete increments, corresponding to the distance between adjacent carrier engaging housing lugs 118 along the longitudinal direction. Interaction of the actuator 120 and positive stops associated with the housing 110, carrier arms (e.g., ratchet slide engaging arm 146 and housing engaging arm 148), and lugs (e.g., carrier engaging housing lugs 118 and carrier engaging ratchet lugs 136) may also combine to give a user tactile and audible feedback as the carrier 140 is incrementally displaced. Further, one or more opening in the housing 110 may allow a user to observe the relative position of the carrier 140, providing further feedback as to carrier 140 position.

As detailed below, the relative position of the carrier 140 with respect to the housing 110 may correlate to the degree of deployment of a stent from the deployment device 100. Thus, visual, audible, and tactile feedback as to the position of the carrier 140 provides a user with information regarding stent deployment during use of the deployment device 100. This information may correlate to increased control during deployment as the practitioner quickly and intuitively can surmise the degree of stent deployment.

As outlined above, tactile and/or audible feedback result from the interactions of the carrier 140, the ratchet slide 130, the housing 110, and/or the actuator 120. For example, as the ratchet slide engaging arm 146 or housing engaging arm 148 of the carrier 140 deflects radially inward then return outward, there may be an audible and/or tactile response.

The device may be configured for visual feedback of, or relating to, the relative deployment of a stent. For example, in some embodiments, the housing 110 may comprise viewing windows to allow a practitioner to observe the position of the carrier 140 relative to the housing 110. Further, indicia on the housing 110 may correlate the position of the carrier 140 to the degree of deployment of a stent.

The increments of displacement of the carrier 140 may correlate to standard stent lengths or units of measure. For example, many stents are sized in 1 cm increments. Configuration of the increments of displacement on the carrier 140 in 1 cm increments would thus directly correlate with stent length at a 1:1 ratio. Any other ratio, including embodiments wherein a stroke correlates to a greater length (such as 2, 3, 4, or 5 cm) or a lesser length (such as 0.01, 0.1, 0.25, 0.5, or 0.75 cm), is likewise within the scope of this disclosure.

In some embodiments, interaction between the carrier 140, the ratchet slide 130, the housing 110, and/or the actuator 120 may comprise additional carrier engaging ratchet lugs 136 and/or carrier engaging housing lugs 118. For example, the carrier engaging ratchet lugs 136 may be spaced to enable semi-continuous ratcheting of the ratchet slide 130 with respect to the actuator 120 and/or the housing 110.

The deployment device 100 may be configured as a universal device operable with various stent lengths. In some embodiments a practitioner may directly equate the number of strokes needed to deploy a stent with the length of the stent loaded in the deployment device 100 (such as four strokes for a four centimeter stent). Further, a single design of deployment device 100 may be utilized with various lengths of stents, with a maximum length related to the maximum length of travel of the carrier 140.

The nature of depression of the actuator 120 may facilitate one-handed operation and may be ergonomically designed. First, a practitioner need only grip the deployment device 100 with one hand to depress the actuator 120, leaving a second hand free for other therapy needs. Further, the direction with which the deployment device 100 is gripped, with the practitioner's hand extending laterally away from the longitudinal axis of the deployment device 100 and the lateral direction of depression, as opposed, for example, to longitudinal gripping to actuate, may be ergonomically desirable. Lateral gripping and input may more readily present the deployment device 100 for use when the delivery catheter assembly 104 is disposed within a patient's body, not requiring the practitioner to move to an awkward stance with respect to other therapy tools. Further, the input portion 121 of the actuator 120 may provide additional surface for a practitioner to grip, facilitating use of a greater portion of a practitioner's hand for actuation, as compared to a finger trigger or similar actuation mechanism.

The incremental displacement of the carrier 140 may further facilitate partial deployment of a stent, allowing a practitioner to deploy the stent in increments, potentially adjusting or confirming the position of the stent between these increments.

Still further, the deployment device 100 may be configured for use with either the right or left hand, or gripped with the fingers or palm in contact with the actuator 120 without changing the design of the deployment device 100. These features may further increase user comfort and control. Viewing windows in the housing 110 to confirm the position on the carrier 140 may be located on one or both sides of the housing 110 and may be associated with indicia correlating to stent length or other factors.

Moreover, the relative lengths of the input portion 121 and the transfer arm 123 of the actuator 120 may be configured to provide mechanical advantage when deploying a stent. This may increase comfort and control during use. The ratio of the length of the input portion 121—from its distal end to the pin aperture 122—to the length of the transfer arm 123—from the pin aperture 122 to the ratchet slide engaging portion 124—may be greater than or equal to 1.5:1, including 2:1, 2.5:1, 3:1, 3.5:1, or greater. This ratio correlates to the mechanical advantage provided by the device. In some instances the mechanical advantage provided may be 1.5:1, 2:1, 2.5:1, 3:1, 3.5:1, or greater. Stated another way, the ratio of length of travel of the input portion 121 to the corresponding length of travel of the ratchet slide engaging portion 124 may be 1.5:1, 2:1, 2.5:1, 3:1, 3.5:1, or greater. Accordingly, the input force applied against the input portion 121 may result in a greater force exerted by the ratchet slide engaging portion 124 on the ratchet slide 130. The ratio of the force exerted on the ratchet slide 130 to the input force may be 1.5:1, 2:1, 2.5:1, 3:1, 3.5:1, or greater.

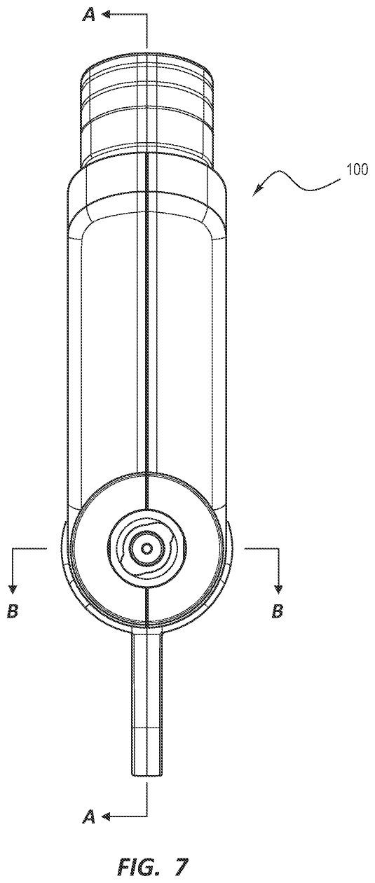

FIG. 7 is a front view of the deployment device 100, illustrating two cross-sectional planes. Specifically, plane A-A extends vertically along the longitudinal axis of the deployment device 100 viewing the exposed components in a right to left direction. Plane A-A corresponds to the cross-sectional plane of FIGS. 2, 3B, and 5. Plane B-B also extends from the longitudinal axis of the deployment device 100, though Plane B-B extends horizontally therefrom. Plane B-B corresponds to the cross-sectional plane of FIG. 6, and is viewed from a top to bottom direction. The longitudinal axis of the deployment device 100 is in both planes A-A and B-B, with the line defined as the intersection between these planes being the same line as the longitudinal axis as referenced herein.

Additionally, as stated above, the deployment device 100 may comprise a safety member 180. FIG. 8 is a perspective view of the safety member 180 of the deployment device 100. The safety member 180 may be configured with a circular or partially circular opening configured to snap onto an outside surface of a portion of the deployment device 100. Referring to both FIG. 2 and FIG. 8, the safety member 180 may comprise a safety lug 189 that extends through a ratchet slide safety opening (139 of FIG. 3A) and a similar safety opening in the housing 110 (not shown). When the safety lug 189 is disposed within these openings, the safety lug 189 may prevent proximal displacement of the carrier 140 and the ratchet slide 130, thus preventing inadvertent deployment of a stent. A practitioner may leave the safety member 180 in place during displacement of the delivery catheter assembly 104 to a treatment region. Due to interactions between the carrier 140, the ratchet slide 130, and the actuator 120, the safety member 180 likewise prevents displacement of the actuator 120 when the safety lug 189 extends through the openings.

In the depicted embodiment, the safety lug 189 extends through a bottom portion of the housing 110 and the ratchet slide 130. In other embodiments, the safety lug 189 may extend through a top surface of the housing 110, interacting with the carrier 140 but not directly with the ratchet slide 130. Nevertheless, prevention of proximal displacement on the carrier 140 only will also prevent displacement of the ratchet slide 130 and the actuator 120 due to the interaction between these elements.

In some embodiments, the safety member 180 may be tethered to the deployment device 100 or may comprise a sliding switch or other element operably coupled to the housing 110 or other components of the deployment device 100. In the depicted embodiment, the safety member 180 is removably coupled.

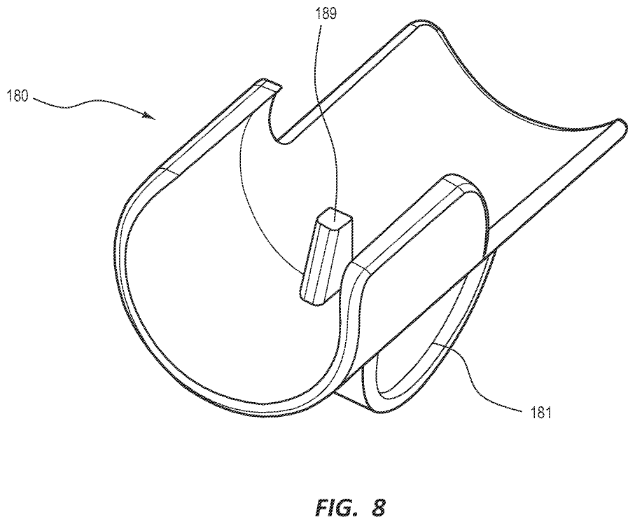

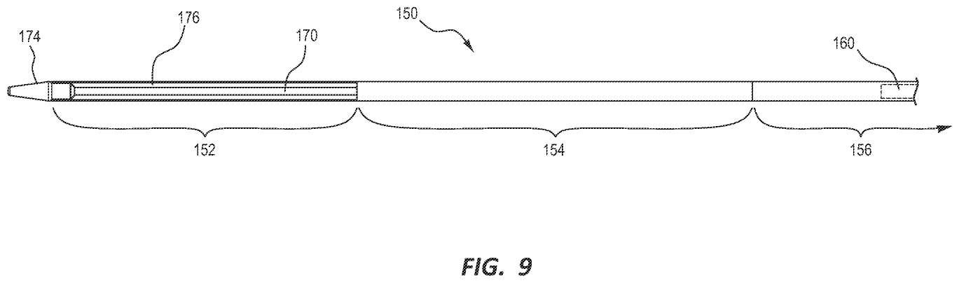

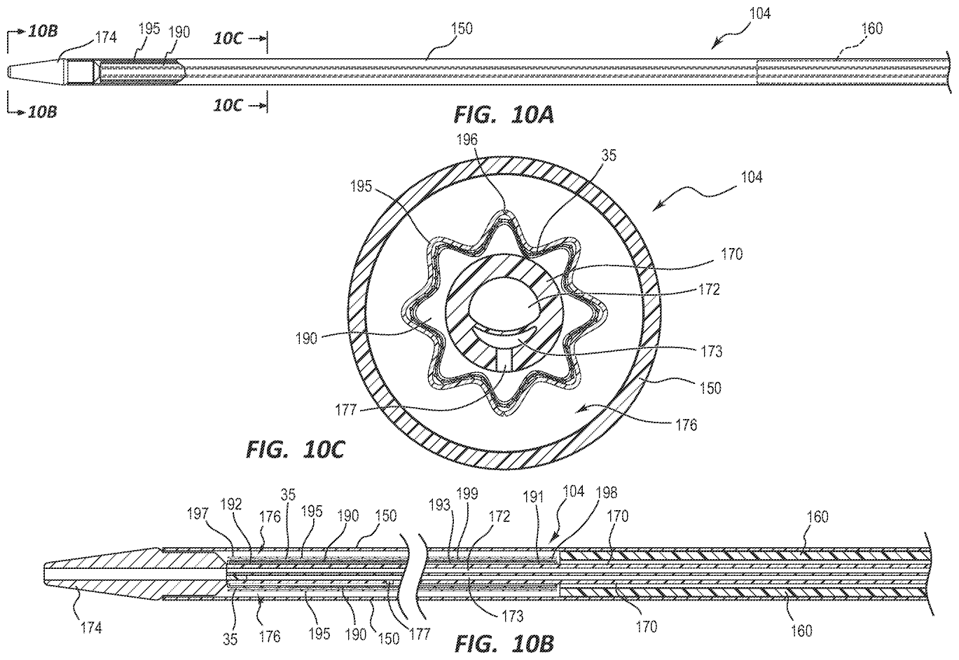

FIG. 9 is a side view of a distal portion of the delivery catheter assembly 104 of the deployment device 100. FIG. 10A is a side view of the same distal portion of the delivery catheter assembly 104 as shown in FIG. 9. FIG. 10B is a cross-sectional view of the distal portion of the delivery catheter assembly 104 through plane 10B-10B. FIG. 10C is a longitudinal cross-sectional view of the distal portion of the delivery catheter assembly 104 through plane 10C-10C

Referring to FIGS. 1, 2, 9, and 10A-10C, the delivery catheter assembly 104 may be configured to deploy a self-expanding stent 35 as the deployment device 100 is manipulated, as discussed above. The delivery catheter assembly 104 may comprise an outer sheath 150, extending from the handle assembly 102. The outer sheath 150 may be fixedly coupled to the carrier 140. The delivery catheter assembly 104 may further comprise an intermediate sheath 160 and an inner sheath 170, both disposed within the outer sheath 150, and both fixedly coupled to the housing 110. Thus, proximal displacement of the carrier 140 with respect to the housing 110 will proximally displace the outer sheath 150 with respect to both the intermediate sheath 160 and the inner sheath 170. In certain embodiments, the self-expanding stent 35 is a bare-metal stent have a self-expanding structure. In other embodiments, the self-expanding stent 35 is a stent graft having a multilayered construct of cover layers and a self-expanding structure.

As illustrated in FIG. 9, the outer sheath 150 may comprise a shaft section 156 extending from the carrier 140 in a distal direction. At the distal end of the shaft section 156 the outer sheath 150 may comprise a flex zone 154 extending from the shaft section 156 in a distal direction. Finally, the outer sheath 150 may comprise a pod 152 extending from the flex zone 154 in a distal direction. (As shown in FIG. 9, the pod 152 may be transparent.)

The shaft section 156 of the outer sheath 150 may have a different stiffness and/or durometer than the flex zone 154 and/or the pod 152. The flexibility toward the distal end of the outer sheath 150 may improve trackability of the delivery catheter assembly 104 over a guidewire and may be less traumatic, while a stiffer shaft may be more kink resistant and/or transmit displacement and/or torque along the shaft section 156. In other words, the stiffer shaft section 156 may allow the practitioner to push, pull, and rotate the delivery catheter assembly 104 as it is advanced through the vasculature of the patient and as the stent is deployed. An outer diameter of the outer sheath 150 may range from about 7 Fr to about 14 Fr.

The pod 152 may be configured to retain a crimped or otherwise constrained stent 35. Removal of the pod 152 from the stent 35 may allow the stent 35 to be partially expanded, such as by self expansion, and thereby deploy. It is within the scope of this disclosure for the pod 152 to be any relative length, the flex zone 154 to be any relative length, and the shaft section 156 to be any relative length. Thus, in some instances, a constrained stent may be in one, two, or all three of these portions of the outer sheath 150. For example, in the illustrated embodiment, an annular space 176 (described further below) is configured to receive the stent 35 in a crimped state extending along the pod 152 as well as portions of the flex zone 154 and the shaft section 156. In other embodiments, the annular space 176 may correlate just to the pod 152 segment, meaning the device is configured to retain a stent only within the pod 152 segment.

The distal tip 174 of the delivery catheter assembly 104 may be coupled to and/or integrally formed with the inner sheath 170. A first lumen 172 may extend along the inner sheath 170 from the proximal end of the deployment device 100 to the distal tip 174. A luer fitting 113 coupled to the housing 110 may be in communication with the first lumen 172. A guidewire may thus extend through the luer fitting 113, through the first lumen 172, and out of the distal tip 174. Further, fluid introduced into the luer fitting 113 may be utilized to flush the first lumen 172. A second lumen 173 may extend along the inner sheath 170 in parallel with the first lumen 172 from the proximal end of the deployment device 100 to the pod 152. A luer fitting 114 coupled to extension tubing 127 coupled to the housing 110 may be in fluid communication with the second lumen 173. Fluid (e.g., air or liquid) introduced into the luer fitting 114 may be utilized to expand a pliant expandable member or balloon 190. A fluid aperture 177 through a wall of the inner sheath 170 may facilitate flow of the fluid from the second lumen 173 to an interior of the balloon 190. In some embodiments, a plurality of fluid apertures 177 may be disposed along a length of the balloon 190 to facilitate flow of the fluid into different portions of the balloon 190.

The inner sheath 170 may be fixed to the housing 110, for example, at the proximal end of the inner sheath 170. An intermediate sheath 160, also fixed to the housing 110, may extend over a portion of the inner sheath 170. The intermediate sheath 160 and the inner sheath 170 may or may not be directly fixed to each other. In some embodiments, the intermediate sheath 160 may be a close slip fit over the inner sheath 170.

The inner sheath 170 can extend distally beyond a distal end of the intermediate sheath 160, creating the annular space 176 between the inner sheath 170 and the outer sheath 150 adjacent the distal tip 174, extending proximally to the distal end of the intermediate sheath 160. This annular space 176 may be configured to contain the stent in the crimped state.

As the deployment device 100 is manipulated to displace the carrier 140 with respect to the housing 110, the outer sheath 150 is displaced proximally with respect to the inner sheath 170 and the intermediate sheath 160. The distal end of the intermediate sheath 160 can interact with the proximal end of the stent 35, preventing the stent 35 from being drawn back with the outer sheath 150.

In some embodiments, a fluid aperture 162 in the intermediate sheath 160 may extend through the wall of the intermediate sheath 160 and the wall of the inner sheath 170, into fluid communication with the first lumen 172. This fluid aperture 162 may thus provide fluid communication between the annular space 176 and the first lumen 172, as fluid within the first lumen 172 can move through the fluid aperture 162 and into the annular space 176. This communication may be used to flush the annular space 176 during use, which may be configured to remove air or other unwanted materials in the annular space 176 or around the stent 35.

The distal tip 174 may comprise a flexible material and may be configured to be atraumatic. The distal tip 174 may comprise nylons, including Pebax® polyether block amides.

In some instances, braided or coil reinforcements may be added to the outer sheath 150, the intermediate sheath 160, and/or the inner sheath 170 to increase kink and/or elongation resistance. Reinforcing members may comprise stainless steel, nitinol, or other materials and may be round, flat, rectangular in cross section, and so forth.

One, two, or all of the outer sheath 150, the intermediate sheath 160, and/or the inner sheath 170 may be configured with varying durometers or other properties along the length thereof. In some instances, the outer sheath 150 may be configured with a proximal section with a durometer between 72 and 100 on the Shore D scale or may be greater than 100 on the Shore D scale. A second portion of the outer sheath 150 may comprise a durometer of 63 on the Shore D scale, and a distal section with a durometer between 40 and 55 on the Shore D scale. Any of these values, or the limits of any of the ranges, may vary by 15 units in either direction. In some instances, the second portion will begin about six inches from the distal end of the outer sheath 150, and the distal section will begin about three inches from the distal end of the outer sheath 150. These sections may or may not correspond to the shaft section 156, the flex zone 154, and the pod 152 as described above. The intermediate sheath 160 may be configured with varying durometer zones within the same ranges of hardness and length.

Any of the inner sheath 170, the intermediate sheath 160, and the outer sheath 150 may have differing durometer or flex zones along their lengths, and these zones may overlap in various ways to create various stress/strain profiles for the overall delivery catheter assembly 104. Overlapping of such zones may reduce a tendency to kink, including a tendency to kink at transition zones. Further, the housing 110 may be coupled to a strain relief member 116 (as shown in FIG. 2).

Any of the outer sheath 150, the intermediate sheath 160, and the inner sheath 170 may be comprised of nylons, including Pebax® polyether block amides. Further, during manufacture, any of these members may be configured with a low friction outer surface, including through “frosting” the materials, by blowing air across the material during extrusion, or by using additives during extrusion to reduce friction.

In some instances, during manufacture the distal tip 174 may be pulled into interference with the outer sheath 150, prestressing the inner sheath 170 in tension. This may reduce any effects of material creep or elongation during sterilization, keeping the distal tip 174 snugly nested with the outer sheath 150. Further, during manufacture, the interface zone between the outer sheath 150 and the carrier 140 may be configured with a tolerance zone, meaning the outer sheath 150 can be coupled to the carrier 140 at multiple points along an inside diameter of the carrier 140. This tolerance may enable manufacturing discrepancies or variations to be taken up during assembly to ensure a snug nest between the distal tip 174 and the outer sheath 150. The same tolerance fit may be applied to the inner sheath 170 and/or the intermediate sheath 160 wherein these members couple to the housing 110, including a fit zone along an inside diameter of the luer fitting 113.

In some instances, the outer sheath 150 may include indicia correlating to the degree to which the stent 35 has been deployed. These indicia may correspond to the position of the outer sheath 150 with respect to the housing 110. For example, as the outer sheath 150 is drawn into the housing 110, different indicia are exposed and/or covered.

In certain embodiments, the outer sheath 150 may include a lubricious coating to reduce the coefficient of friction. The lubricious coating may facilitate smooth passage of the delivery catheter assembly 104 through a hemostasis valve of an introducer sheath if used during insertion of the delivery catheter assembly 104, through a percutaneous insertion site of a patient, or through a tortuous vasculature of a patient. The lubricious coating may cover an outer surface of the outer sheath 150 from a proximal end to a distal end. In other embodiments, the lubricious coating may cover a portion of the outer sheath 150. For example, the lubricious coating may cover a distal portion of the outer sheath 150. The lubricious coating can include any suitable material. For example, in some embodiments, the lubricious coating may include a hydrophilic fluid activated material, such as polyvinylpyrrolidone, polyvinylpyrrolidone and polyurethane blend, hyaluronic acid, etc. In other embodiments, the lubricious coating may include silicone oil. In still other embodiments, the lubricious coating may include a dry lubricant, such as parylene, methylvinylether/maleic anhydride (i.e., Gantrez), polyvinylpyrrolidone acrylic acid copolymers, perfluoropolyethers and other fluorinated polymers, dimethyl acrylamide-glycidyl methacrylate copolymer, etc. The lubricious coating can be applied to the outer sheath 150 using any suitable technique, such as dipping, spraying, deposition, wiping, etc. In certain embodiments, the intermediate sheath 160 and/or the inner sheath 170 may include the lubricious coating.

Additionally or alternatively, the outer sheath 150 may be configured for use without an introducer sheath. That is, in some embodiments, a lubricous coating applied to the outer sheath 150 may facilitate direct advancement of the delivery catheter assembly 104 into the body. For example, in some procedures, a practitioner may access a body lumen via a needle and/or guidewire. The catheter assembly 104 may be advanced through the skin and into the lumen (along a wire or without a wire) such that the outer sheath 150 is in direct contact with the patient's tissue, including the wall of the lumen. A lubricous coating may facilitate sealing of the lumen wall against the outer sheath 150 to prevent or minimize fluid leakage, such as leakage of blood from a vascular lumen.

Furthermore, in embodiments wherein the catheter assembly 104 is configured for use without an introducer sheath, catheter assembly 104 may be configured with sufficient rigidity along its length to facilitate pushing the catheter assembly 104 into the body, while a distal portion of the catheter assembly 104 is sufficiently flexible to facilitate tracking along a wire. For example, the material properties (such as durometer) of the outer sheath 150, the intermediate sheath 160, and/or the inner sheath 170 may be configured to facilitate pushability and trackability for sheathless use. As noted above the material properties of each of these elements may vary along their lengths. Additionally, as discussed above, overlapping or offsetting flex zones in any of these elements may reduce kinking and, in turn, enhance pushability of the catheter assembly 104.

Regardless of whether an introducer sheath is used, in some instances, the deployment device 100 may be configured such that the outer sheath 150 may be distally displaced after the stent 35 is deployed to nest the distal tip 174 in the outer sheath 150 during withdrawal of the deployment device 100 from a patient. Such configurations may include features of the handle assembly 102 that disengage the carrier 140 from one or more elements after stent deployment.

Referring to FIGS. 10A-10C, the delivery catheter assembly 104 may comprise the outer sheath 150. The delivery catheter assembly 104 may further comprise the intermediate sheath 160 and the inner sheath 170, each of which can be disposed within the outer sheath 150. Additionally, the inner sheath 170 can be disposed within the intermediate sheath 160. In certain embodiments, the delivery catheter assembly 104 may lack the intermediate sheath 160. In some embodiments, the outer sheath 150 may be displaced with respect to each of the intermediate sheath 160 and the inner sheath 170.

The annular space 176 may be disposed between each of the outer sheath 150 and the inner sheath 170. In certain embodiments, the annular space 176, or a portion of the annular space 176, may be configured to receive and/or retain the stent 35 in the crimped state. Removal or displacement of the outer sheath 150 from around the stent 35 may allow the stent 35 to be expanded, and thereby deploy. It is within the scope of this disclosure for the annular space 176 to be any relative length. Thus, in some instances, the stent 35 may be disposed along only a portion of a length of the annular space 176. In some other instances, the stent 35 may be disposed along substantially the entire length of the annular space 176.

In various embodiments, the intermediate sheath 160 may be directly coupled to the inner sheath 170. In various other embodiments, the intermediate sheath 160 may not be directly coupled to the inner sheath 170. For example, the intermediate sheath 160 may be a close slip fit over the inner sheath 170.

As depicted, the inner sheath 170 can extend distally beyond a distal end of the intermediate sheath 160, creating or forming the annular space 176 between the inner sheath 170 and the outer sheath 150 adjacent the distal tip 174. Furthermore, the annular space 176 may extend proximally from adjacent the distal tip 174 to adjacent the distal end of the intermediate sheath 160. The annular space 176 may be configured to retain the stent 35.

The balloon 190 may be coupled to and surround or be disposed around the inner sheath 170. As shown, the balloon 190 may be disposed around a circumference of the inner sheath 170. For example, distal and proximal end portions of the balloon 190 may be coupled to a portion of an exterior surface of the inner sheath 170. The balloon 190 may also be disposed within a portion of the annular space 176. In some embodiments, the balloon 190 may be configured to engage and/or retain the stent 35 as the outer sheath 150 is withdrawn proximally. Stated another way, the balloon 190 may at least partially grip, anchor, hold, and/or grasp the stent 35. In certain embodiments, the stent 35 may be disposed around the balloon 190 and then the stent 35 may be constrained, crimped, and/or loaded around the balloon 190. Further, a portion of the stent 35 (e.g., an inner surface of the stent 35) when loaded on the balloon 190 may imprint within a portion of the balloon 190 (e.g., an outer surface of the balloon 190) as discussed in further detail below.

In some embodiments, the balloon 190 may comprise a thermoplastic elastomer (e.g., ChronoPrene™). A portion of the inner sheath 170 may be formed from a polyether block amide (e.g., Pebax®), and the ChronoPrene™ can couple with or form a bond with (e.g., a strong bond with) the Pebax® inner sheath 170. In other embodiments, the balloon 190 can comprise one or more silicones, polyether block amides (e.g., Pebax®), other thermoplastic elastomers, and/or other suitable materials. In certain embodiments, the balloon 190 may be formed from multiple materials (e.g., the balloon 190 may include two or more layers). The balloon 190 may be coupled to the inner sheath 170 using adhesives, fasteners, RFID welding, melt bonding, and so forth. Other suitable methods of coupling the balloon 190 onto a surface (e.g., a surface of the inner sheath 170) are also within the scope of this disclosure.

In some embodiments, the durometer of the balloon 190 may be about 10 to about 60 on the Shore A scale, about 15 to about 45 on the Shore A scale, about 20 to about 30 on the Shore A scale, about 23 to about 27 on the Shore A scale, or another suitable durometer. In some other embodiments, the durometer of the balloon 190 may be about 25 on the Shore A scale. In other embodiments, the durometer of the balloon 190 may vary over the length of the balloon 190.

In some embodiments, the balloon 190 may be configured to limit or prevent longitudinal displacement of the stent 35. For example, when the stent 35 is crimping within the deployment pod, the unexpanded balloon 190 may grip the stent 35 such that longitudinal displacement of the stent 35 is limited or prevented. In certain embodiments, the balloon 190 may be configured to limit or prevent the stent 35 from collapsing or accordioning (e.g., longitudinally folding on itself) during deployment (for example as the outer sheath 150 is retracted, the balloon 190 can maintain the longitudinal position of the stent 35). For example, the balloon 190 may provide axial support to the stent 35. Further, the balloon 190 may be configured to partially surround one or more portions of the stent 35, meaning that the balloon 190 may conform to at least a portion of the stent 35. For example, the balloon 190 may conform to portions of the inner surface, shape, edges, and/or texture of the stent 35.

The stent 35 (e.g., the inner surface of the stent 35) may at least partially imprint around the balloon 190. In some embodiments, the stent 35 is a helical stent (e.g., a stent having a helical stent geometry). Imprinting of the helical stent around the balloon 190 may support rows of coils of the helical stent. Imprinting of the helical stent around the balloon 190 may support each row of coils of the helical stent. In some other embodiments, imprinting of a non-helical stent (e.g., a stent having a non-helical stent geometry) around the balloon 190 may support rows of coils of the non-helical stent. Imprinting of the non-helical stent around the balloon 190 may support each row of coils of the non-helical stent.

In certain embodiments, the presence of the balloon 190 may increase the force needed to proximally displace or pull back on the outer sheath 150. For example, disposition of the balloon 190 and/or the stent 35 within the annular space 176 may cause or form a tighter fit between each of the inner sheath 170 and the outer sheath 150. However, due at least in part to the mechanical advantage that can be provided by the handle assembly 102, as discussed above, the stent 35 can still be readily deployable by a user.

As illustrated, the balloon 190 can extend longitudinally along a portion of the inner sheath 170 and/or through a portion of the annular space 176. The balloon 190 may have varying lengths. In some embodiments, the balloon 190 may extend from adjacent a proximal end of the distal tip 174 to a position adjacent the distal end of the intermediate sheath 160. In some other embodiments, the balloon 190 may extend along only a portion of a longitudinal distance between each of the proximal end of the distal tip 174 and the distal end of the intermediate sheath 160. As depicted, the distal end of the intermediate sheath 160 can be disposed proximally of the balloon 190.

The delivery catheter assembly 104 may be configured to receive and/or retain the stent 35 having varying lengths. In various embodiments, the balloon 190 may have a length that is greater than a length of the stent 35. In various other embodiments, the balloon 190 may have a length that is substantially equal to the length of the stent 35. In various other embodiments, the balloon 190 may have a length that is less than the length of the stent 35.

In some embodiments, the balloon 190 can be longitudinally continuous along the length of the stent 35. For example, the balloon 190 may extend longitudinally along the entire length of the stent 35. In certain embodiments, the balloon 190 can be circumferentially continuous along an inside surface of the stent 35. For example, the balloon 190 may extend along the entire inner circumference of the stent 35.

The balloon 190 may also include a range of wall thicknesses (e.g., the distance from an interior surface of the balloon 190 to an exterior surface of the balloon 190). In certain embodiments, the wall thickness of the balloon 190 may be from about 0.0005 inch to about 0.050 inch, including from about 0.001 inch to about 0.050 inch, or another suitable thickness.

In some embodiments, a compound or drug may be loaded in the balloon 190 and/or on an outer surface of the balloon 190. For example, an anticoagulant drug or an anti-cell proliferation drug may be loaded in and/or coated on the balloon 190.

The balloon 190 can be radially expandable to a diameter ranging from about 1 millimeter to about 16 millimeters. In some embodiments, the balloon 190 may be expandable over its entire length substantially simultaneously. In other embodiments, the balloon 190 can comprise a proximal portion 191, a distal portion 192, and an intermediate portion 193. Each of the portions 191, 192, 193 can be configured to be radially expandable at different pressures such that they can be expanded sequentially or in stages. This can be accomplished by varying the material durometer, stiffness, thickness, reinforcements, etc. of the wall of the balloon 190 over the proximal, distal, and intermediate portions 191, 192, 193, respectively. For example, the proximal portion 191 can include a thin wall and be configured to expand at a low pressure, the intermediate portion 193 can include a wall thicker than the wall of the proximal portion 191 and be configured to expand at a moderate pressure, and the distal portion 192 can include a wall thicker than the wall of the intermediate portion 193 and be configured to expand at high pressure resulting in sequential or staged expansion of the balloon 190 from the proximal portion 191 to the distal portion 192 as the balloon 190 is pressurized. Other expansion sequences are contemplated, such as the distal portion 192 prior to the proximal portion 191 and the intermediate portion 193, the intermediate portion 193 prior to the proximal portion 191 and the distal portion 192, and the proximal portion 191 and the distal portion 192 prior to the intermediate portion 193.

As illustrated in the embodiment of FIGS. 10A-10C, the delivery catheter assembly 104 can include a sleeve 195 configured to retain the stent 35 in a crimped state and to be ruptured by the balloon 190 when radially expanded. As shown, the sleeve 195 can be disposed over the stent 35 such that the sleeve 195 circumferentially surrounds the stent 35 from a proximal end to a distal end. In other embodiments, the sleeve 195 may surround a portion of the length of the stent 35 such as a proximal portion, a distal portion, an intermediate portion, or any combination thereof. The sleeve 195 may be formed from of any suitable biocompatible, semi-rigid material, such as polyethylene, polypropylene, polystyrene, and so forth. Other materials are contemplated.

The sleeve 195 can include a weakened portion 196 configured to rupture when the balloon 190 radially expands the sleeve 195. In some embodiments, the sleeve 195 can include more than one weakened portions 196 circumferentially disposed around the sleeve 195. For example, the sleeve 195 may include two, three, four, or more weakened portions 196. In certain embodiments, the weakened portion 196 has variable strength along the length of the sleeve 195 such that the sleeve 195 may be ruptured sequentially or in stages. For example, a distal portion 197 of the sleeve 195 may include the weakened portion 196 having a rupture strength weaker than a proximal portion 198 and/or an intermediate portion 199 such that the distal portion 197 ruptures prior to the proximal portion 198 and/or the intermediate portion 199. Other staged ruptures of the sleeve 195 are contemplated. In some embodiments, the weakened portion 196 may include a plurality of perforations in a wall of the sleeve 195 disposed longitudinally along the length of the sleeve 195. The plurality of perforations can be spaced closer together within one or more of the portions 197, 198, 199 than in one or more of the other portions 197, 198, 199 to facilitate rupture of the closer perforations prior to the other perforations. In another embodiment, the weakened portion 196 may include a variable thickness wall where a thinner wall is rupturable prior to a thicker wall.

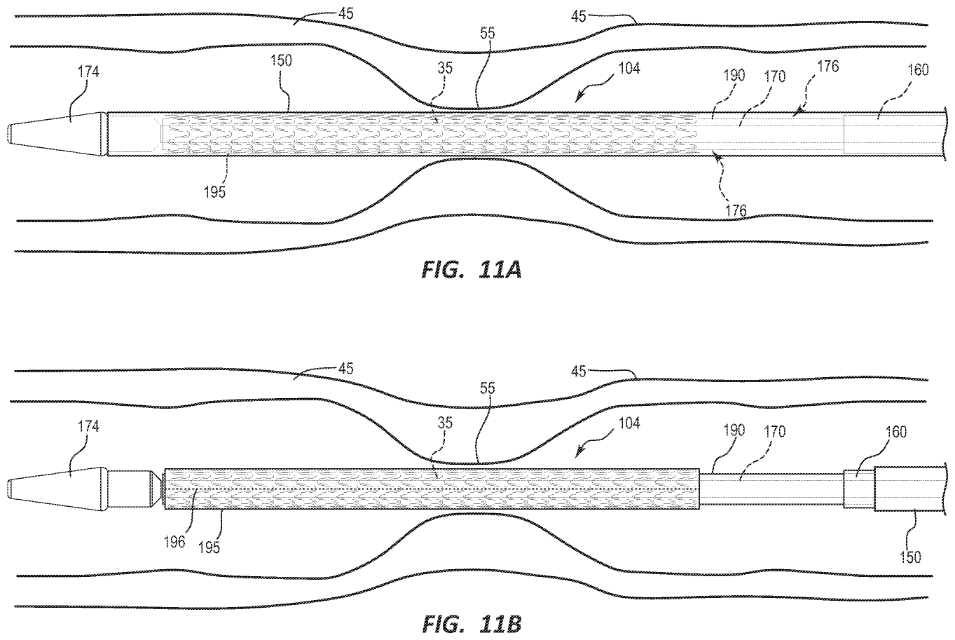

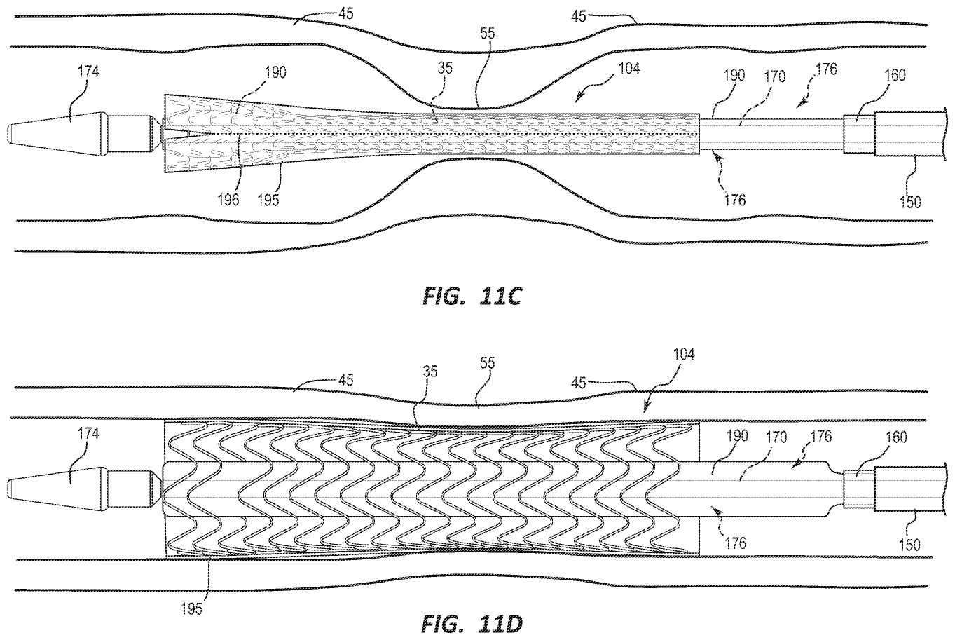

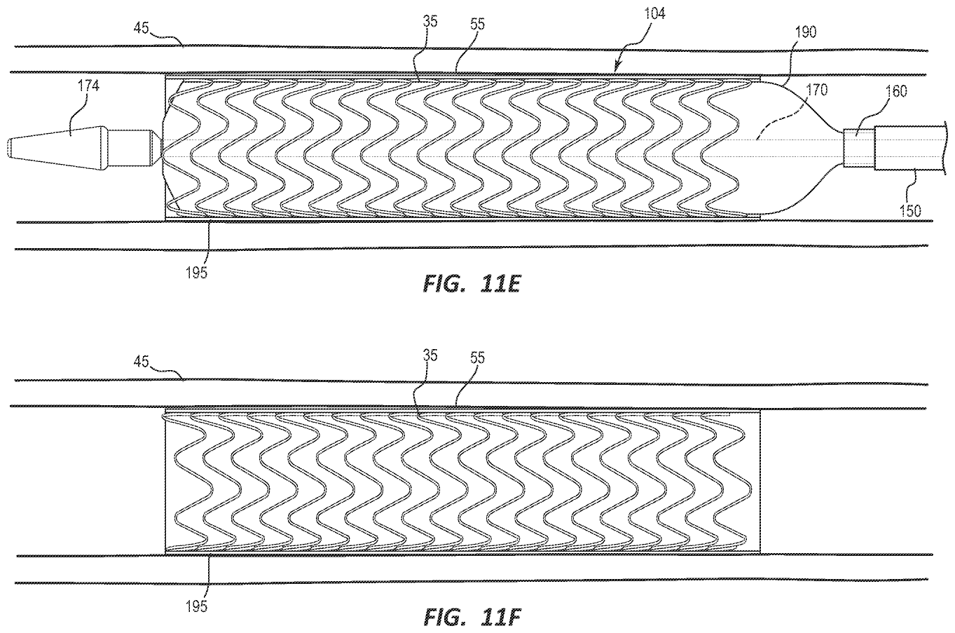

FIGS. 11A-11E are side views of the distal portion of the delivery catheter assembly 104 in a first state, a second state, a third state, a fourth state, and a fifth state, respectively. FIG. 11F is a side view of a deployed self-expanding stent 35.

With reference to FIG. 11A, the self-expanding stent 35 may be crimped or disposed around the balloon 190 within the annular space 176. As illustrated, the distal portion of the delivery catheter assembly 104 can be disposed within a vessel 45 (e.g., a vessel of a patient) adjacent a lesion 55 (e.g., narrowing of the vessel 45) to be treated. In the first state, as illustrated, the outer sheath 150 may be disposed over the stent 35. The stent 35 can extend from the proximal end of the distal tip 174 along only a portion of the balloon 190, such that a gap or space is present along the balloon 190 (e.g., between a proximal end of the stent 35 and the distal end of the intermediate sheath 160). In some embodiments, the stent 35 may extend along substantially an entire length of the balloon 190. In some other embodiments, the stent 35 may be longer than the balloon 190. For example, in some instances, only a portion of the stent 35 is disposed over the balloon 190.

FIG. 11B depicts the distal portion of the delivery catheter assembly 104 in the second state. To deploy the stent 35, the outer sheath 150 may be displaced proximally in relationship to the stent 35, the intermediate sheath 160, the inner sheath 170, and/or the balloon 190. In some embodiments, the outer sheath 150, the intermediate sheath 160, and/or the inner sheath 170 may be operably coupled to the actuator 120, as discussed above in reference to the deployment device 100. In some other embodiments, the outer sheath 150, the intermediate sheath 160, and/or the inner sheath 170 may be operably coupled to the housing 110, as discussed above in reference to the deployment device 100, and the housing 110 may be operably coupled to the actuator 120.

Furthermore, displacement of the actuator 120 may be configured to displace the outer sheath 150 relative to the stent 35, the inner sheath 170, the balloon 190, and/or the intermediate sheath 160. As noted above, some embodiments of the delivery catheter assembly 104 may lack an intermediate sheath 160.

In certain embodiments, as noted above, the deployment device 100 and/or the actuator 120 may be configured to withdraw the outer sheath 150 in a continuous motion to expose an entire length of the stent 35. In other embodiments, as noted above, the deployment device 100 and/or the actuator 120 may be configured to incrementally withdraw the outer sheath 150 to incrementally expose the stent 35. For example, the outer sheath 150 may be configured to be proximally displaced relative to the inner sheath 170, the balloon 190, and the stent 35 in a step-wise or incremental manner.

In certain embodiments, the balloon 190 may grip or support a constrained portion of the stent 35 such that the stent 35 is restricted from longitudinal proximal displacement by the outer sheath 150 as the outer sheath 150 is withdrawn proximally over the stent 35. This can result in improved accuracy of placement of the stent 35 at the lesion 55. In other embodiments that include the intermediate sheath 160, the stent 35 can abut the distal end of the intermediate sheath 160 to prevent proximal displacement of the stent when the outer sheath 150 is withdrawn.

FIG. 11C depicts the delivery catheter assembly 104 in the third state, wherein deployment of the stent 35 can be initiated by partial radial expansion of the balloon 190. The balloon 190 can be radially expanded by injection of air or liquid through the luer fitting 114, the extension tubing 127, the second lumen 173 of the inner sheath 170, the fluid aperture 177, and into the balloon 190. As noted above, the balloon 190 can be expanded uniformly along its length or in a staged expansion.

When the balloon 190 is partially expanded, the weakened portion 196 of the sleeve 195 can be ruptured. As illustrated in FIG. 11C, a distal portion 197 of the sleeve 195 can be ruptured prior to rupture of the proximal portion 198 and the intermediate portion 199. In other embodiments, as noted above, the weakened portion 196 can be ruptured substantially simultaneously over its length or in a staged rupturing process as the balloon 190 is partially expanded.

FIG. 11D depicts the delivery catheter assembly 104 in the fourth state, wherein the sleeve 195 may be fully ruptured to alloy the stent 35 to deploy from the delivery catheter assembly 104 and self-expand such that a radial outward compressive force is applied to the lesion 55 by the stent 35 to radially expand a diameter of the lesion 55. The ruptured sleeve 195 may be disposed between the stent 35 and the wall of the vessel In other embodiments, the sleeve 195 may be removed when the delivery catheter assembly 104 is withdrawn from the vessel 45.

FIG. 11E depicts the delivery catheter assembly 104 in the fifth state, wherein the balloon 190 is substantially fully expanded. When expanded, the balloon 190 can apply a radial outward compressive force to the stent 35 to further expand the diameter of the lesion 55 and/or to smooth out the stent 35 such that wrinkles in the stent 35 are substantially removed.

FIG. 11F depicts the stent 35 fully expanded within the vessel 45 such that the diameter of the lesion 55 is substantially equivalent to the diameter of the vessel 45. The delivery catheter assembly 104 can be withdrawn from the vessel 45. There may not a need to further insert a balloon catheter to further expand the stent 35 and smooth wrinkles from the stent 35.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of deploying a self-expanding stent may include one or more of the following steps: positioning a distal portion of stent deployment catheter assembly adjacent a vascular target to be treated; withdrawing an outer sheath of the stent deployment catheter assembly relative to a self-expanding stent to expose the self-expanding stent; partially expanding an expandable member of the stent deployment catheter assembly; rupturing a sleeve surrounding the self-expanding stent; allowing the self-expanding stent to self-expand to apply a radial outward directed force to the vascular target, wherein a diameter of the vascular target is expanded; and expanding the expandable member to radially expand the self-expanding stent and smooth wrinkles from the self-expanding stent. Other steps are also contemplated.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use.

“Fluid” is used in its broadest sense, to refer to any fluid, including both liquids and gases as well as solutions, compounds, suspensions, etc., which generally behave as fluids.

References to approximations are made throughout this specification, such as by use of the terms “substantially” or “about.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially the entire length” is recited with respect to a feature, it is understood that in further embodiments, the feature can have a precisely entire length configuration.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A stent delivery catheter assembly, comprising:
an outer sheath;
an inner sheath co-axially disposed within the outer sheath;
an expandable member surrounding a distal portion of the inner sheath; a
self-expanding stent surrounding the expandable member; and
a sleeve surrounding and selectively constraining the self-expanding stent, wherein the expandable member is configured to rupture the sleeve, allowing the self-expanding stent to radially self-expand.

2. The stent delivery catheter assembly of claim 1, wherein a length of the expandable member is equal to or greater than a length of the self-expanding stent.

3. The stent delivery catheter assembly of claim 1, wherein a distal portion of the expandable member is configured to radially expand before a proximal portion and an intermediate portion.

4. The stent delivery catheter assembly of claim 1, wherein the inner sheath comprises:
a first lumen extending therethrough; and
a second lumen extending therethrough and in fluid communication with the expandable member.

5. The stent delivery catheter assembly of claim 1, wherein the self-expanding stent is configured to imprint around a portion of an outer surface of the expandable member when the self-expanding stent is in a crimped state.

6. The stent delivery catheter assembly of claim 5, wherein the expandable member is configured to limit longitudinal displacement of the self-expanding stent when the outer sheath is proximally withdrawn.

7. The stent delivery catheter assembly of claim 1, wherein the sleeve comprises a weakened portion longitudinally disposed in a wall of the sleeve.

8. The stent delivery catheter assembly of claim 7, wherein the weakened portion is configured to allow the expandable member to rupture a distal portion of the sleeve before an intermediate portion and a proximal portion of the sleeve.

9. The stent delivery catheter assembly of claim 1, wherein the sleeve surrounds a length of the self-expanding stent from a proximal end to a distal end of the self-expanding stent.

10. A method of deploying a self-expanding stent, comprising:
positioning a distal portion of stent deployment catheter assembly adjacent a vascular target to be treated;
withdrawing an outer sheath of the stent deployment catheter assembly relative to a self-expanding stent to expose the self-expanding stent;
partially expanding an expandable member of the stent deployment catheter assembly;
rupturing a sleeve surrounding the self-expanding stent;
allowing the self-expanding stent to self-expand to apply a radial outward directed force to the vascular target, wherein a diameter of the vascular target is expanded; and
expanding the expandable member to radially expand the self-expanding stent and smooth wrinkles from the self-expanding stent.

11. The method of claim 10, wherein partially expanding the self-expanding stent comprises radially expanding a distal portion of the expandable member before radially expanding a proximal portion and an intermediate portion of the expandable member.

12. The method of claim 10, wherein partially expanding the self-expanding stent comprises radially expanding a proximal portion of the expandable member before radially expanding a distal portion and an intermediate portion of the expandable member.

13. The method of claim 10, wherein partially expanding the self-expanding stent comprises radially expanding an intermediate portion of the expandable member before radially expanding a proximal portion and a distal portion of the expandable member.

14. The method of claim 10, wherein partially expanding the self-expanding stent comprises radially expanding a distal portion and a proximal portion of the expandable member before radially expanding an intermediate portion of the expandable member.

15. The method of claim 10, wherein rupturing the sleeve comprises rupturing a weakened portion of the sleeve.

16. The method of claim 15, wherein rupturing the weakened portion of the sleeve comprises rupturing a distal portion of the sleeve before rupturing an intermediate portion and a proximal portion of the sleeve.

17. A prosthesis delivery catheter assembly, comprising:
an outer sheath;
an inner sheath co-axially disposed within the outer sheath; and
an expandable pliant member surrounding a distal portion of the inner sheath, wherein the expandable pliant member is configured to engage a self-expanding prosthesis.

18. The prosthesis delivery catheter assembly of claim 17, wherein the self-expanding prosthesis is configured to imprint around a portion of an outer surface of the expandable pliant member.

19. The prosthesis delivery catheter assembly of claim 17, wherein a length of the expandable pliant member is equal to or less than a length of the self-expanding prosthesis.

20. The prosthesis delivery catheter assembly of claim 17, wherein the expandable pliant member limits longitudinal displacement of the self-expanding prosthesis.
